(19) United States
(12) Patent Application Publication
(10) Pub. No.: US 2026/0307319 A1
(43) Pub. Date: Oct. 8, 2026

(54) EXTRUDABLE FIBER COMPOSITE MATERIAL AND ITS USE AND PRODUCTION

(71) Applicant: No Plastic Trading AG, Cham (CH)

(72) Inventors: Lina GRABER, Wetzikon (CH); Beat GRÜTER, Ebikon (CH); Simon HOFER, Ellikon an der Thur (CH); Guy PETIGNAT, Erlenbach (CH)

(21) Appl. No.: 19/481,678

(22) PCT Filed: May 8, 2024
(86) PCT No.: PCT/EP2024/062755

§ 371 Date: Nov. 5, 2025

(30) Foreign Application Priority Data
May 9, 2023 (CH) 000495/2023

(51) Int. Cl. B29C 48/06; A61F 13/38; B29C 48/09; B29C 48/40; B29K 201/00

(52) U.S. Cl. CPC B29C 48/06; A61F 13/38; B29C 48/09; B29C 48/40; B29K 2003/00; B29K 2201/00

(57) ABSTRACT

The present invention relates to an extrudable fiber composite material, a rod-shaped body extruded therefrom, including its use as a disposable article, and a corresponding production process.

The present invention relates to an extrudable fiber composite material, a rod-shaped body extruded therefrom, including its use as a disposable article, and a corresponding production process.

Common disposable articles are still produced cost-effectively from plastics which are not biodegradable and pollute the environment. Known plastic alternatives are sometimes comparatively expensive and complicated to produce or have unfavorable properties for disposable articles.

The object of the present invention was to provide a material which is biodegradable and is suitable for producing, inter alia, disposable articles.

This object is achieved according to the invention by an extrudable fiber composite material according to claim 1.

The extrudable fiber composite material according to the invention contains:
25-80% by weight, optionally 25-60% by weight, optionally 30-40% by weight or 55-80% by weight of starch,
10-65% by weight of cellulose, in particular 10-34% by weight of cellulose or 35-65% by weight of cellulose, and
at least 1% by weight of water to 100% by weight.

It has surprisingly been found that the extrudable fiber composite material according to the invention has a sufficiently high dimensional stability and at the same time is sufficiently flexible to substantially prevent a breakage of objects produced therefrom with corresponding (in particular the intended) manipulation.

For all aspects and embodiments of the present invention, “disposable article” is understood to mean both articles which are disposed of after single use and after repeated use. However, the repeated use is numerically restricted, for example to a 2- to 5-fold use.

The specification of the percentages by weight, for example 10-65% by weight, means 10% by weight to 65% by weight.

For all aspects and embodiments of the present invention, “extrusion” is understood to mean processes in which plastically deformable to thick viscous masses are pressed out under pressure, for example from a shaping opening/environment. The extrusion can be carried out in a piston extruder or a screw extruder, in particular a single-screw or twin-screw extruder.

The extrudable fiber composite material according to the invention is biodegradable and consists of environmentally compatible materials. Furthermore, the fiber composite material can be processed by means of extrusion to various shapes and objects.

In one embodiment, the extrudable fiber composite material according to the invention can be thermoplastic.

According to all aspects, the starch for use in the fiber composite material according to the invention can be any known and/or commercially available starch, in particular thermoplastic starch, semi-thermoplastic starch or starch blends. In one example, the fiber composite material according to the invention contains 30-40% by weight of starch, in particular 30-35% by weight of starch, in particular 30% by weight of starch.

According to all aspects, the cellulose for use in the fiber composite material according to the invention can be any known and/or commercially available cellulose and can be present or used in the form of fibers, for example cellulose fibers of cotton, bast, wheat fibers, wood fibers, flax or waste paper (including recycled paper). Furthermore, the cellulose for use in the fiber composite material according to the invention can be selected from hemicellulose, a cellulose derivative, for example methylcellulose, cellulose acetate, cellulose nitrate, viscose and celluloid.

According to all aspects, the cellulose for use in the fiber composite material according to the invention can be a microcellulose and/or nanocellulose, which can be produced in particular with the aid of microorganisms (for example bacteria and/or fungi).

The fiber composite material according to the invention can contain plasticizers, for example 1-20% by weight, 1-9% by weight, 10-20% by weight or 2-3% by weight, or it can be plasticizer-free, that is to say that no plasticizer is present in the fiber composite material. The plasticizer can be suitable for making the fiber composite material softer, more flexible, more pliable and/or more elastic. Suitable plasticizers are known to the person skilled in the art, non-toxic and environmentally compatible plasticizers being preferred in particular.

The fiber composite material produced in this way is, for example, compostable, biodegradable, at the same time and has, for example, good resistance to high atmospheric humidity with at the same time acceptable mechanical strength. Furthermore, the material can, for example, also be sufficiently elastic and flexible in dry air.

In one embodiment of the extrudable fiber composite material, the cellulose is cellulose fibers, in particular cellulose fibers of cotton.

The extrudable fiber composite material contains the specified components (starch, cellulose and water), wherein further components can optionally be contained as long as the specified % by weight are satisfied. Further components can be, for example, plasticizers, dyes, stabilizers or fillers.

In one embodiment of the extrudable fiber composite material, the fiber composite material contains 1-20% by weight of plasticizer, in particular 1-9% by weight, 2-3% by weight or 10-20% by weight of plasticizer, in particular selected from acetamide, malic acid, asparagine, ethanolamine, ethylene glycol, formamide, glycerol, urea, ionic liquids, in particular 1-butyl-3-methylimidazolium chloride (BMIM Cl), isoleucine, maltodextrin, sorbitol, xylitol, polyhydric alcohols, in particular octanediol or pentanediol, and a mixture thereof.

Further embodiments of the extrudable fiber composite material result from the features of dependent claim 3.

In one embodiment of the extrudable fiber composite material, the cellulose has a fiber length of 10 mm or less, in particular 5 mm or less, in particular 2 mm or less, in particular a fiber length of 30-90 μm.

Cellulose fibers can be obtained, for example, as waste product of cotton processing. The use of these cellulose fibers proves to be cost-effective since it is a waste product.

Further embodiments of the extrudable fiber composite material result from the features of dependent claims 4 and 5.

In one embodiment of the extrudable fiber composite material, the cellulose consists of cellulose fibers of different lengths, in particular long and short cellulose fibers. In one embodiment of the invention, the long cellulose fibers have a fiber length of 10 mm to 5 mm and the short cellulose fibers have a fiber length of less than 5 mm, in particular 2 mm or less, in particular a fiber length of 30-90 μm. The use of short cellulose fibers having a size in the μm and/or nm range is possible (corresponding to microcellulose and/or nanocellulose).

Long fibers have the property of producing mechanical strength in the product, short fibers promote both crystallization of the starch and at the same time produce beautiful, smooth surfaces.

In one embodiment of the extrudable fiber composite material, the cellulose consists of cellulose fibers of different lengths and the extrudable fiber composite material contains 1-20% of plasticizer, in particular 1-20% by weight, 1-9% by weight or 10-20% by weight of plasticizer, in particular selected from polyhydric alcohols, in particular octanediol or pentanediol, and a mixture thereof.

The invention further relates to a rod-shaped body according to claim 6.

The rod-shaped body according to the invention can be a solid body (without cavity) or a hollow body.

The rod-shaped body according to the invention can in particular be an extruded rod-shaped body and contains an extrudable fiber composite material containing:
starch,
cellulose, in particular with a fiber length of 10 mm or less, in particular 5 mm or less, in particular 2 mm or less, in particular with a fiber length of 30-90 μm, and
water.

The definitions and terms cited in connection with the extrudable fiber composite material also apply to the rod-shaped body.

The extrudable fiber composite material for the rod-shaped body can be thermoplastic. The fiber composite material of the rod-shaped body can contain plasticizer, for example 1-20% by weight, 1-9% by weight, 10-20% by weight or 2-3% by weight, or it can be plasticizer-free, that is to say that no plasticizer is present in the fiber composite material.

The rod-shaped body can be a hollow body and/or can have a round (including oval) or polygonal cross section and can be of any desired length. The term “rod-shaped” means in this connection that the length of the body is greater than the diameter. The body can be a hollow body and have a hollow profile in one embodiment, the hollow body being continuously hollow substantially along its entire length.

The rod-shaped body contains the specified components (starch, cellulose and water), wherein further components can optionally be contained. Further components can be, for example, dyes, plasticizers, stabilizers or fillers.

Embodiments of the rod-shaped body result from the features of dependent claims 7 to 11. In one embodiment of the rod-shaped body, it contains the extrudable fiber composite material containing:
25-80% by weight, optionally 25-60% by weight, optionally 30-40% by weight or 55-80% by weight of starch,
10-65% by weight of cellulose, in particular 10-34% by weight of cellulose or 35-65% by weight of cellulose, and
at least 1% by weight of water to 100% by weight,
   wherein the cellulose optionally has a fiber length of 10 mm or less, in particular 5 mm or less, in particular 2 mm or less, in particular a fiber length of 30-90 μm.

The cellulose fibers can be aligned in the rod-shaped body along its longitudinal axis.

In one example of the rod-shaped body, it contains the extrudable fiber composite material containing 30-40% by weight of starch, in particular 30-35% by weight of starch, in particular 30% by weight of starch.

In one embodiment of the rod-shaped body, the extrudable fiber composite solid further contains plasticizer, in particular 1-20% by weight, 1-9% by weight, 2-3% by weight or 10-20% by weight of plasticizer, in particular selected from acetamide, malic acid, asparagine, ethanolamine, ethylene glycol, formamide, glycerol, urea, ionic liquids, in particular 1-butyl-3-methylimidazolium chloride (BMIM Cl), isoleucine, maltodextrin, sorbitol, xylitol, polyhydric alcohols, in particular octanediol or pentanediol, and a mixture thereof.

In one embodiment of the rod-shaped body, the body has a diameter of 2 to 15 mm, in particular 2 to 10 mm, in particular 2 to 3 mm, in particular approximately 2.5 mm.

In one embodiment, the rod-shaped body is a hollow body, in particular a hollow body having a wall thickness of 0.3 to 4 mm, in particular 0.3 to 0.7 mm, in particular 0.35 to 0.55 mm, advantageously approximately 0.45 mm.

It has surprisingly been found that the rod-shaped body, in particular the hollow body, having the aforementioned dimensions and comprising the extrudable fiber composite material according to the invention is dimensionally stable, but does not tend to break, but only to buckle (for example during intended use). As a result, a safe use of the rod-shaped body, in particular hollow body, for example for hygiene products, cosmetic articles, food carriers, in particular for handle bodies for suckers (lollipops) or ice cream, or for drinking straws, can be ensured.

In one embodiment of the rod-shaped body, in particular hollow body, it is coated with a layer which is impermeable to water and/or water vapor, in particular wax from the food industry, in particular beeswaxes, gum mastic, carnauba waxes or shellac.

The layer which is impermeable to water and/or water vapor has the effect, for example, that the body, in particular hollow body, does not become brittle in an environment with low atmospheric humidity, since the moisture (that is to say the water) remains enclosed in the extrudable fiber composite material.

The invention further relates to a cotton swab according to claim 12 and the use of the rod-shaped body, in particular hollow body, according to claim 13.

The cotton swab according to the invention has the rod-shaped body, in particular hollow body, as handle body and a cotton body arranged at at least one free end of the handle body.

The handle body of the cotton swab can have, for example, a length of 2 to 10 cm, in particular a length of 5 to 10 cm. The handle body can have a diameter of 2 to 3 mm, in particular approximately 2.5 mm, and, as hollow body, a wall thickness of 0.3 to 4 mm, in particular 0.3 to 0.7 mm, in particular 0.35 to 0.55 mm, advantageously approximately 0.45 mm.

The use according to the invention relates to the use of the rod-shaped body, in particular hollow body, as a disposable article, in particular as a hygiene product, as a cosmetic article, in particular as a handle body for a cotton swab, as a food carrier, in particular as a handle body for suckers (lollipop) or for ice cream, or as a drinking straw.

The invention further relates to a process according to one of claims 14 or 15.

The process according to the invention for producing the rod-shaped body, in particular hollow body, comprises the following steps:
(a) mixing the cellulose, the water and the starch, and
(b) extruding the mixture from step (a) to form a rod-shaped body, in particular hollow body. In one embodiment of the process according to the invention, the components from step (a) additionally comprise a plasticizer, so that the cellulose, the water, the plasticizer and the starch are mixed in step (a). Step (a) can be carried out in any suitable mixing apparatus which comprises, for example, an extrusion device or is spatially and/or functionally assigned to such an extrusion device. The extrusion device can further comprise an extruder or have an extruder spatially and/or functionally assigned.

Further optional components of the rod-shaped body, in particular hollow body, for example dyes, can be added in step (a).

Embodiments of the process result from the features of dependent claim 16.

In one embodiment of the process according to the invention, a single-screw or twin-screw extruder is used for the extrusion in step (b).

In order to produce the cavity in the hollow body, the extrusion mass (that is to say the mixture in step (b) which emerges from the extruder) can be passed through a channel which comprises, arranged centrally, a separating body which produces the cavity in the hollow body. The separating body can further comprise, in the extrusion direction, a nozzle through which a gas (for example air) is blown which prevents the cavity in the hollow body from closing after passing through the separating body.

The process according to the invention can in particular be a continuous process in which the rod-shaped body or hollow body is extruded as an endless longitudinal body.

Exemplary embodiments of the present invention will be explained in more detail below with reference to figures.

Shown are:

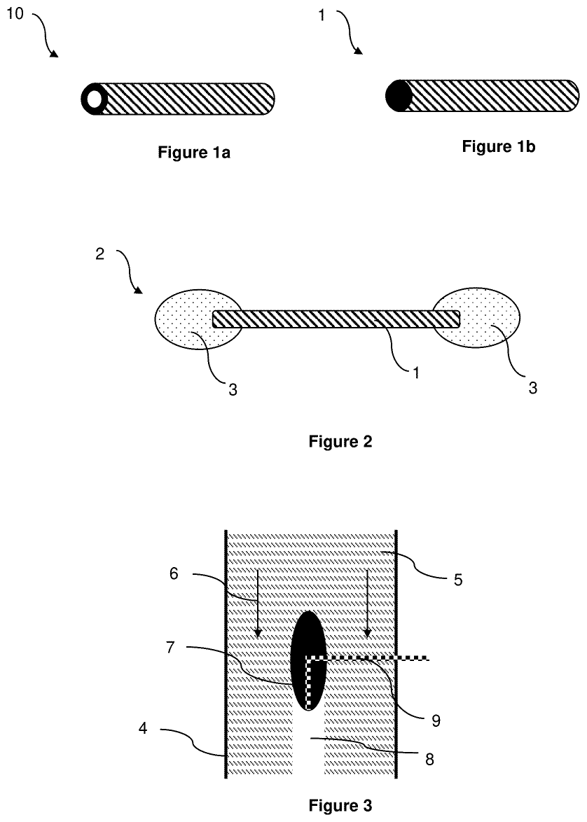

FIG. 1a a rod-shaped hollow body;

FIG. 1b a rod-shaped body (solid body);

FIG. 2 a cotton swab; and

FIG. 3 an extrusion channel with separating body.

FIG. 1a shows the rod-shaped hollow body (10) and FIG. 1b the rod-shaped solid body (1). A cavity (FIG. 1a) extends in the hollow body along its length or longitudinal axis (from left to right in FIG. 1a).

FIG. 2 shows a cotton swab (2) with the rod-shaped body or hollow body (1) as handle body and the cotton bodies (3).

FIG. 3 shows an exemplary extrusion channel (4) through which the extrusion mass (5) (that is to say the mixture in step (b) of the process according to the invention which emerges from the extruder) moves in the direction of the arrow (6), for example in a process according to the invention for producing the rod-shaped hollow body. A separating body (7) in the extrusion channel (4) produces a cavity (8) in the extrusion mass (5). In order to prevent the cavity (8) from closing in the extrusion direction (6) after the separating body (7), the nozzle (9) is aerated actively or passively with a gas (for example air).

1. An extrudable fiber composite material containing:
25-80% by weight of starch,
10-65% by weight of cellulose, and
at least 1% by weight of water to 100% by weight.

2. The extrudable fiber composite material according to claim 1, further containing 1-20% of a plasticizer, wherein the plasticizer is selected from at least one of acetamide, malic acid, asparagine, ethanolamine, ethylene glycol, formamide, glycerol, urea, ionic liquid, 1-butyl-3-methylimidazolium chloride (BMIM Cl), isoleucine, maltodextrin, sorbitol, xylitol, polyhydric alcohol, octanediol, pentanediol, and a mixture thereof.

3. The extrudable fiber composite material according to claim 1, wherein the cellulose has a fiber length of 10 mm or less.

4. The extrudable fiber composite material according to claim 1, wherein the cellulose consists of cellulose fibers of different lengths.

5. The extrudable fiber composite material according to claim 1, wherein the cellulose consists of cellulose fibers of different lengths and wherein the extrudable fiber composite material contains 1-20% of plasticizer wherein the plasticizer is selected from at least one of polyhydric alcohol, octanediol, pentanediol, and a mixture thereof.

6. A rod-shaped body or an extruded rod-shaped body containing an extrudable fiber composite material containing:
starch,
cellulose, and
water.

7. The rod-shaped body according to claim 6, wherein the extrudable fiber composite material further contains plasticizer, wherein the plasticizer is selected from at least one of acetamide, malic acid, asparagine, ethanolamine, ethylene glycol, formamide, glycerol, urea, ionic liquids, 1-butyl-3-methylimidazolium chloride (BMIM Cl), isoleucine, maltodextrin, sorbitol, xylitol, polyhydric alcohol, octanediol, pentanediol, and a mixture thereof.

8. The rod-shaped body according to claim 6, containing an extrudable fiber composite material containing 25-80% by weight of starch, 10-65% by weight of cellulose, and at least 1% by weight of water to 100% by weight.

9. The rod-shaped body according to claim 6, wherein the rod-shaped body has a diameter of 2 to 15 mm.

10. The rod-shaped body according to claim 6, wherein the rod-shaped body is a hollow body, in particular a hollow body having a wall thickness of 0.3 to 4 mm.

11. The rod-shaped body according to claim 6, wherein the rod-shaped body is coated with a layer which is impermeable to water and/or water vapor.

12. A cotton swab comprising a rod-shaped body according to claim 6 as handle body and a cotton body arranged at at least one free end of the handle body.

13. The rod-shaped body according to claim 6, wherein the rod-shaped body is a disposable article, a hygiene product, a cosmetic article, a handle body, for a food carrier, or a drinking straw.

14. A process for producing the rod-shaped body according to claim 6, the process comprising the steps:
(a) mixing the cellulose, the water and the starch, and
(b) extruding the mixture from step (a) to form a rod-shaped body or a rod-shaped body that is a hollow body.

15. A process for producing the rod-shaped body according to claim 7, the method comprising the steps:
(a) mixing the cellulose, the water, the plasticizer and the starch, and
(b) extruding the mixture from step (a) to form a rod-shaped body or a rod-shaped body that is a hollow body.

16. The process according to claim 14, wherein a single-screw or twin-screw extruder is used for the extrusion in step (b).

17. The rod-shaped body according to claim 6, wherein the cellulose has a fiber length of 10 mm or less.

18. The rod-shaped body according to claim 11, wherein the layer is a wax from the food industry, beeswax, gum mastic, carnauba wax, or shellac.

19. A cotton swab, a lollipop, or an ice cream comprising the rod-shaped body according to claim 6 as a handle body or a food carrier.
